(19) United States
(12) Patent Application Publication
(10) Pub. No.: US 2025/0352395 A1
(43) Pub. Date: Nov. 20, 2025

(54) ANTIMICROBIAL HEMOSTATIC DEVICES AND METHODS OF USE AND MAKING

(71) Applicant: TELEFLEX LIFE SCIENCES II LLC, WILMINGTON, DE (US)

(72) Inventors: Roland Ostapoff, West Hartford, CT (US); Jessica Gould, Middletown, CT (US); Charles Wing, Milford, CT (US); Oliva Hart, Cheshire, CT (US); Adam Alenckis, Oakville, CT (US); Jocelyn Kurtze, Wallingford, CT (US); Jim Murto, Hanover, MN (US); Tuan Pham, Roseville, MN (US)

(21) Appl. No.: 19/211,685

(22) Filed: May 19, 2025

Related U.S. Application Data
(60) Provisional application No. 63/648,798, filed on May 17, 2024.

(51) Int. Cl. A61F 13/01; A61L 15/18; A61L 15/20; A61L 15/44

(52) U.S. Cl. CPC A61F 13/01017; A61L 15/18; A61L 15/20; A61L 15/44; A61L 2300/104; A61L 2300/106; A61L 2300/206; A61L 2300/606

(57) ABSTRACT

The disclosure relates to an antimicrobial hemostatic device having a substrate configured to be in contact with a bleed, where the substrate includes a hemostatic agent, a biguanide based antimicrobial agent, or a pharmaceutically acceptable salt thereof, and a binder configured to maintain the hemostatic agent with the substrate. The disclosure further includes methods of making and using such devices.

This application claims the benefit of U.S. Provisional Patent Application No. 63/648,798, filed May 17, 2024, the contents of which are incorporated by reference in their entirety as if fully set forth herein.

### TECHNICAL FIELD

The present disclosure relates to hemostatic devices and their methods of use and making. In particular, hemostatic devices with a biguanide based antimicrobial agent.

### BACKGROUND

Death from hemorrhages or bleeds is a substantial global problem. It is estimated that there about 2 million deaths globally per year from hemorrhages, of which, 1.5 million are from physical trauma. (Cannon, J W, Hemorrhagic Shock. New England Journal of Medicine, vol. 378, 4, Jan. 25, 2018, p. 370-379). According to the National Trauma Institute, trauma is the number one cause of death among Americans between the ages of 1 and 46 years. (Latif R K, et al. Traumatic hemorrhage and chain of survival. Scand J Trauma Resusc Emerg Med. 2023 May 24; 31(1):25). However, experts believe that 20% of deaths from exsanguination, loss of blood, could be prevented with fast action to control the bleeding. (news.cornell.edu/stories/2019/03/bleeding-control-basics-taught-cornell-health-sessions).

While there is a need for devices, systems, and methods for controlling blood loss, open wounds may further lead to deadly or crippling infections. For example, the infection rate from wounds can ranges from 5% to 32%, depending on various factors. (Roodsari G S, et al. The risk of wound infection after simple hand laceration. World J Emerg Med. 2015; 6(1):44-7).

Adequately managing both blood loss and risk of infection following trauma is not trivial. By way of example, it is recognized that not all components which can be used to manage bleeds or infection are compatible with each other. For instance, U.S. Pat. No. 5,980,925 states: it is taught that chlorhexidine and its derivatives are inhibited by a variety of ingredients including anionic surfactants, soaps, gums, sodium alginate, magnesium aluminum silicate, magnesium trisilicate, bentonite, talc, kaolin, high pH, 3% lecithin/polysorbate 80 and polysorbate: 80. (Interaction between Cosmetic Ingredients and Preservatives, COSMETICS & TOILETRIES 110:81-86 (1995)). Accordingly, there remains a need for devices, systems, and methods which control both blood loss and infection.

### SUMMARY

The disclosure relates to an antimicrobial hemostatic device. In particular to an antimicrobial hemostatic device having a substrate configured to be in contact with a bleed, where the substrate includes a hemostatic agent, a biguanide based antimicrobial agent, or a pharmaceutically acceptable salt thereof, and a binder configured to maintain the hemostatic agent with the substrate. The disclosure further includes methods of making and using such devices.

### BRIEF DESCRIPTION OF THE DRAWINGS

FIG. 1 shows a graph of clot times for various medical devices.

FIG. 2 shows a graph of clot times for various medical devices.

FIG. 3 shows a graph of clot times for various medical devices.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to

include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

As used herein, a “biguanide based antimicrobial agent” refers to compounds having —C(NH2)(NH—)C(NH2)(NH2)—) in their chemical structure and act as antimicrobial agents against bacteria and other microorganisms.

Examples of bisbiguanide based antimicrobial agents include: chlorhexidine, polyhexamethylene biguanide (PHMB), alexidine, isononabutidine, nonabutidine, octihexidine, heptoctidine, hexidecidine, heptihexidine, hexoctidine, hexhexidine, chlorhexidine. (Octenidine, polyhexamethylene biguanide (PHMB), Propamidine isethionate, hexamidine, polyhexanide, acridine-based bisbiguanides)

As used herein, a “a hemostatic agent” refers to a material that helps stop bleeding. Including through mechanically sealing the bleeding site, actively accelerating the clotting cascade, or concentrating clotting factors, preferably actively accelerating the clotting cascade, in most preferred embodiments the hemostatic agent has a surface charge which accelerates the clotting cascade. Examples of hemostatic agents include: alumino silicates (such as kaolin, bentonite, and halloysites among others), chitosan, thrombin, among others.

As used herein, a “binder” refers to a material used to adhere the hemostatic agent to the substrate preventing excess loss of the hemostatic agent in the packaging and prior to application. This can be reversible or irreversible. Examples of binders include: glycerin, polyethylene glycol, polyvinyl alcohol, polypropylene glycol, other polyols and chitosan.

As used herein, a “substrate” refers to a physical support material that the active ingredient can be adhered to. Examples of substrates include textiles such gauze, preferably polyester-rayon gauze, or cotton/cellulose, or sponge, or a rigid gel.

As used herein, a “medical device” refers to any device which treats wounds. In preferred embodiments, the medical device is a gauze itself.

A device of the disclosure can include one or more anti-microbial or anti-bacterial components in addition to the biguanide based antimicrobial agent. As used herein, anti-microbial or anti-bacterial components refers to a material or compound used to reduce microbial growth and kill microbes.

Examples of such components include silver based components such as: colloidal silver, silver chloride, silver sulfadiazine, or silver nitrate. Other components include iodine compounds such as povidone-iodine or cadexomer iodine.

In addition to being described by various structural components, a device of the disclosure can be defined by its properties. For example, a device within the present disclosure can have a clot time of less than 180 seconds and preferably less than less than 150 seconds. In some embodiments, the device has a clot time between 100 and 150 seconds.

A device of the disclosure can further be defined by its antimicrobial properties. For example, a device of the disclosure has a 6 or 4 log reduction relative to a control.

### EXAMPLES

### Example 1

Gauze was cut into 0.5″ by 0.5″ squares. Uncoated gauze samples were weighed and moisture content were recorded for each sample. Samples were placed in test tubes and the test tubes labeled.

Beakers of deionized (DI) water, kaolin, and glycerin were prepared according to Table 1.

TABLE 1

| | Target Kaolin | | | |
|---|---|---|---|---|
| | Concentration | Amount of | Amount of | Volume of DI |
| Test | (μg/cm²) | Kaolin (mg) | Glycerin (mg) | water (mL) |
| 1 | 64.5 | 10.4 | 27.8 | 13 |
| 2 | 129.0 | 20.8 | 55.6 | 13 |
| 3 | 258.1 | 41.6 | 111.1 | 13 |
| 4 | 516.1 | 83.2 | 222.2 | 13 |
| 5 | 1032.2 | 166.5 | 444.5 | 13 |
| 6 | 2064.5 | 333.0 | 888.9 | 13 |
| 7 | 4129.0 | 665.8 | 1777.9 | 13 |

Beakers containing the mixtures were placed on a magnetic stirrer with a stirring rod placed therein. The stir plate was set to 300 RPM. Once the mixture appeared uniform, 130 μL of the mixture was pipetted into each of the appropriately labeled tubes. 20 samples per concentration were used. The samples were tested in a Tilt Tube Clot Test method described below.

A water bath was prepared to 37±1° C., and a test tube holder was placed therein. One mL of whole sheep blood at 37° C. was pipetted into each test tube. 150 μL of CaCl₂ at 37° C. was then pipetted into a tube and a timer was then started (time “0”). Tubes were capped and gently shaken twice to ensure that the CaCl₂ was completely mixed with the blood, and then placed in the water bath. Tubes were tilted by 90° at 15 second intervals, beginning when the timer reads 45 seconds. The time to clot was determined by visual inspection and the clot time was recorded. A clot is defined as a solid mass that may or may not adhere to bottom of test tube.

The average clot time for control blood and hemostatic agents was then calculated. The results are shown in Table 2. The test would be disregarded if the control did not clot between 7 and 14 minutes.

TABLE 2

| Variable | Test | Total Count | Mean | StDev | Minimum | Median | Maximum |
|---|---|---|---|---|---|---|---|
| Lag-Adjusted | 1 | 20 | 153.85 | 30.10 | 120.00 | 146.25 | 251.75 |
| Clot Time (s) | 2 | 20 | 125.35 | 13.32 | 101.50 | 131.25 | 161.25 |
| | 3 | 20 | 116.95 | 6.48 | 105.00 | 116.75 | 131.75 |
| | 4 | 20 | 109.75 | 10.05 | 98.00 | 106.00 | 132.00 |
| | 5 | 20 | 98.75 | 8.89 | 75.00 | 99.88 | 113.00 |
| | 6 | 20 | 93.88 | 7.87 | 78.75 | 92.38 | 112.50 |
| | 7 | 20 | 88.50 | 10.21 | 71.25 | 86.25 | 101.25 |

The results illustrate that the clotting benefits associated with increasing kaolin concentration above 1000 μg/cm² provides diminishing improvements.

### Example 2

To make the substrate, gauze was cut into 4″+/−0.5″ by 6″+/−0.5″ rectangles. The uncoated gauze samples were then dried and the weight and moisture content taken.

Next, three coating mixtures were prepared according to Table 3.

TABLE 3

| Test | Kaolin (g) | Glycerin (g) | Water (mL) |
|---|---|---|---|
| 8 | 8.58 | 22.92 | 300 |
| 9 | 8.58 | 22.92 | 375 |
| 10 | 8.58 | 22.92 | 450 |

Once mixture appeared uniform, the mixture was poured into a coating vessel to cover the bottom (˜20 mL), and the gauze was dipped into mixture and lifted to let drip while ensuring the gauze was completely saturated.

The coated gauze was placed on wire rack and placed in an 80° C. oven for 24 hours to dry. Samples were then weighed, and the moisture content was recorded. Uncoated dry weight, coated dry weight, and coat weight were calculated. Results are summarized in Table 4.

TABLE 4

| Average weight (mg) | Test 8 | Test 9 | Test 10 |
|---|---|---|---|
| Avg Coat wt. | 606 | 504 | 344 |
| stdev | 75 | 92 | 54 |
| Avg finished good wt. | 1311 | 1209 | 1011 |
| stdev | 98 | 151 | 106 |

Based on the composition of the mixtures for each test (Table 4) and the observed amount of coating mixture the samples absorbed (12 mL on average) the expected/theoretical coat weight (Table 5) was calculated and compared to observed values.

TABLE 5

| Theoretical values | | | |
|---|---|---|---|
| per 4″ by 6″ | | | |
| sample (mg) | Test 8 | Test 9 | Test 10 |
| Kaolin | 343 | 275 | 229 |
| Glycerin | 917 | 733 | 611 |
| Coat weight | 1260 | 1008 | 840 |

Percent (%) error was calculated to compare the theoretical coat weight to the observed coat weight using the following formula:

$$\% error = \frac{theoretical - experimental}{theoretical} \times 100$$

The absolute value was not taken to show the direction of the change. Table 6 shows the average percent error for each test.

TABLE 6

| | Test 8 | Test 9 | Test 10 |
|---|---|---|---|
| Average % error | −52% | −50% | −59% |

Given the error in yield, additional experimentation was undertaken. Ultimately, it was determined that only Test 10 met the parameters of Table 3, and the samples from Test 8 and Test 9 were discarded.

### Example 3

Gauze was cut into 4″+/−0.5″ by 6″+/−0.5″ rectangles. The samples were dried and then the weight and moisture content were measured. Coating mixtures were prepared according to Table 7.

TABLE 7

| Test | Kaolin (g) | Glycerin (g) | CHX* (g) | DI Water (mL) |
|---|---|---|---|---|
| 11 | 1.91 | 5.09 | 0.39¹ | 100 |
| 12 | 1.91 | 5.09 | 0.062¹ | 100 |
| 13 | 1.91 | 5.09 | 0.39² | 100 |

*CHX = Chlorhexidine
¹Chlorhexidine diacetate (CHA)
²Chlorhexidine dihydrochloride (CHD)

The targeted amounts of kaolin and glycerin were 516 μg/cm² and 1394 μg/cm², and the targeted chlorhexidine concentrations were 300 μg/cm² for Test 11 and Test 13 and 50 μg/cm² for Test 12.

To coat the gauze, 20 mL of the coating mixture was poured into a flatbottom glass dish and then the gauze dipped into the mixture. The gauze was then lifted and allowed to drip. The coated gauze was placed on the wire rack and dried at 80° C. for 24 hrs. After drying was complete, samples were taken from the oven and remeasured for weight and moisture.

During the coating process, it was observed that the CHD salt did not dissolve in the DI water. After coating, there were noticeable white clumps on the gauze. After drying, the CHA samples appeared discolored, turning a yellow/orange upon exposure to 80° C. for 24 hours.

Pre-coating and post coating measurements were taken of each sample to analyze how much coating remained on the gauze and percent error was calculated as discussed in Example 2. The coating results are shown in Table 8.

TABLE 8

| | coat wt. | % error coat |
|---|---|---|
| Sample | (mg) | weight |
| 11-1 | 439 | −46% |
| 11-2 | 485 | −45% |
| 11-3 | 426 | −48% |
| 11-4 | 531 | −49% |
| 13-1 | 491 | −45% |
| 13-2 | 452 | −49% |
| 13-3 | 472 | −49% |
| 13-4 | 496 | −44% |
| 12-1 | 425 | −50% |
| 12-2 | 322 | −62% |
| 12-3 | 415 | −51% |
| 12-4 | 402 | −53% |

Samples from Tests 11-13 and Test 10 from Example 2 were then further separated into 0.5″ by 0.5″ squares and added to test tubes. A Tilt Tube Clot Test, as described in Example 1, was performed. The results are summarized in Table 9.

TABLE 9

| | Sub- | Recorded | Recorded | First to Last | Lag-Adjusted |
|---|---|---|---|---|---|
| | group | Clot Time | Clot time | Lag Time | Clot Time |
| Test | Order | (min:sec) | (sec) | (sec) | (sec) |
| Control | A | 12:30 | 750 | 15 | 750 |
| Control | B | 11:30 | 690 | 15 | 686 |
| Control | C | 13:30 | 810 | 15 | 803 |
| Control | D | 12:15 | 735 | 15 | 724 |
| Control | E | 9:30 | 570 | 15 | 555 |
| | | | | Average= | 704 |
| 10-1 | A | 2:00 | 120 | 15 | 120 |
| 10-1 | B | 2:00 | 120 | 15 | 116 |
| 10-1 | C | 2:00 | 120 | 15 | 113 |
| 10-1 | D | 2:15 | 135 | 15 | 124 |
| 10-1 | E | 2:15 | 135 | 15 | 120 |
| | | | | Average= | 119 |
| 10-2 | A | 2:00 | 120 | 15 | 120 |
| 10-2 | B | 1:45 | 105 | 15 | 101 |
| 10-2 | C | 1:45 | 105 | 15 | 98 |
| 10-2 | D | 2:00 | 120 | 15 | 109 |
| 10-2 | E | 3:00 | 180 | 15 | 165 |
| | | | | Average= | 119 |
| 10-3 | A | 2:00 | 120 | 13 | 120 |
| 10-3 | B | 1:45 | 105 | 13 | 102 |
| 10-3 | C | 2:15 | 135 | 13 | 129 |
| 10-3 | D | 2:00 | 120 | 13 | 110 |
| 10-3 | E | 2:00 | 120 | 13 | 107 |
| | | | | Average= | 114 |
| 10-4 | A | 1:45 | 105 | 16 | 105 |
| 10-4 | B | 1:45 | 105 | 16 | 101 |
| 10-4 | C | 1:45 | 105 | 16 | 97 |
| 10-4 | D | 2:00 | 120 | 16 | 108 |
| 10-4 | E | 2:00 | 120 | 16 | 104 |
| | | | | Average= | 103 |
| 11-1 | A | 16:00 | 960 | 15 | 960 |
| 11-1 | B | 16:00 | 960 | 15 | 956 |
| 11-1 | C | 16:00 | 960 | 15 | 953 |
| 11-1 | D | 16:00 | 960 | 15 | 949 |
| 11-1 | E | 9:00 | 540 | 15 | 525 |
| | | | | Average= | 869 |
| 11-2 | A | 24:00 | 1440 | 15 | 1440 |
| 11-2 | B | 24:00 | 1440 | 15 | 1436 |
| 11-2 | C | 11:15 | 675 | 15 | 668 |
| 11-2 | D | 16:30 | 990 | 15 | 979 |
| 11-2 | E | 17:30 | 1050 | 15 | 1035 |
| | | | | Average= | 1112 |
| 11-3 | A | 10:45 | 645 | 15 | 645 |
| 11-3 | B | 24:00 | 1440 | 15 | 1436 |
| 11-3 | C | 24:00 | 1440 | 15 | 1433 |
| 11-3 | D | 13:30 | 810 | 15 | 799 |
| 11-3 | E | 13:00 | 780 | 15 | 765 |
| | | | | Average= | 1016 |
| 11-4 | A | 16:00 | 960 | | 960 |
| 11-4 | B | 16:00 | 960 | | 960 |
| 11-4 | C | 16:00 | 960 | | 960 |
| 11-4 | D | 16:00 | 960 | | 960 |
| 11-4 | E | 16:00 | 960 | | 960 |
| | | | | Average= | 960 |
| 13-1 | A | 2:15 | 135 | 12 | 135 |
| 13-1 | B | 2:15 | 135 | 12 | 132 |
| 13-1 | C | 2:15 | 135 | 12 | 129 |
| 13-1 | D | 2:30 | 150 | 12 | 141 |
| 13-1 | E | 2:30 | 150 | 12 | 138 |
| | | | | Average= | 135 |
| 13-2 | A | 2:30 | 150 | 15 | 150 |
| 13-2 | B | 2:45 | 165 | 15 | 161 |
| 13-2 | C | 2:45 | 165 | 15 | 158 |
| 13-2 | D | 2:45 | 165 | 15 | 154 |
| 13-2 | E | 3:00 | 180 | 15 | 165 |
| | | | | Average= | 158 |
| 13-3 | A | 2:15 | 135 | 15 | 135 |
| 13-3 | B | 2:30 | 150 | 15 | 146 |
| 13-3 | C | 2:30 | 150 | 15 | 143 |
| 13-3 | D | 2:30 | 150 | 15 | 139 |
| 13-3 | E | 2:45 | 165 | 15 | 150 |
| | | | | Average= | 143 |
| 13-4 | A | 2:15 | 135 | 15 | 135 |
| 13-4 | B | 2:30 | 150 | 15 | 146 |
| 13-4 | C | 2:30 | 150 | 15 | 146 |
| 13-4 | D | 2:30 | 150 | 15 | 146 |
| 13-4 | E | 3:00 | 180 | 15 | 176 |
| | | | | Average= | 150 |
| 12-1 | A | 4:00 | 240 | 13 | 240 |
| 12-1 | B | 4:00 | 240 | 13 | 237 |
| 12-1 | C | 4:00 | 240 | 13 | 237 |
| 12-1 | D | 3:45 | 225 | 13 | 222 |
| 12-1 | E | 4:45 | 285 | 13 | 282 |
| | | | | Average= | 243 |
| 12-2 | A | 3:30 | 210 | 12 | 210 |
| 12-2 | B | 3:45 | 225 | 12 | 222 |
| 12-2 | C | 3:30 | 210 | 12 | 207 |
| 12-2 | D | 3:30 | 210 | 12 | 207 |
| 12-2 | E | 3:45 | 225 | 12 | 222 |
| | | | | Average= | 214 |
| 12-3 | A | 3:15 | 195 | 16 | 195 |
| 12-3 | B | 4:00 | 240 | 16 | 236 |
| 12-3 | C | 3:30 | 210 | 16 | 206 |
| 12-3 | D | 4:00 | 240 | 16 | 236 |
| 12-3 | E | 4:00 | 240 | 16 | 236 |
| | | | | Average= | 222 |
| 12-4 | A | 3:15 | 195 | 15 | 195 |
| 12-4 | B | 3:00 | 180 | 15 | 176 |
| 12-4 | C | 3:30 | 210 | 15 | 206 |
| 12-4 | D | 3:45 | 225 | 15 | 221 |
| 12-4 | E | 3:45 | 225 | 15 | 221 |
| | | | | Average= | 204 |

Clot test results show that the tested concentrations for CHA significantly impacted time to clot and did failed to provide clotting at less than 150 seconds. CHID at an estimated 300 μg/cm² did provide clotting at less than 150 seconds at some individual data points.

### Example 4

Using the procedures outlined in Example 2 and Example 3, substrates were prepared using mixtures the mixtures described in Table 10.

TABLE 10

| | | | | DI Water |
|---|---|---|---|---|
| Test | Kaolin (g) | Glycerin(g) | CHX* (g) | (mL) |
| 14 | 2.60 | 6.40 | 1.51² | 302 |
| 15 | 2.60 | 6.40 | 0.755² | 302 |
| 16 | 2.60 | 6.40 | 0.252² | 302 |
| 17 | 2.60 | 6.40 | 0.116¹ | 302 |
| 18 | 2.60 | 6.40 | 0.100¹ | 302 |
| 19 | 2.60 | 6.40 | 0.051¹ | 302 |
| 20 | 2.60 | 6.40 | 0 | 302 |

*CHX = Chlorhexidine
¹Chlorhexidine diacetate (CHA)
²Chlorhexidine dihydrochloride (CHD)

The concentration of kaolin was targeted at 670.95 μg/cm²; CHD concentrations were targeted at 300, 150, and 50 μg/cm²; and CHA concentrations were targeted at 30, 20, and 10 μg/cm².

The coat drying process was modified such that CHD/non-CHX samples were dried at 80 C for 2h, and CHA samples were dried at 54 C for four hours.

After coating was completed, the four samples from each test were sterilized with high dose gamma sterilization in the range of 50-60 kGy and four samples from each test were left non-sterile, i.e., not irradiated.

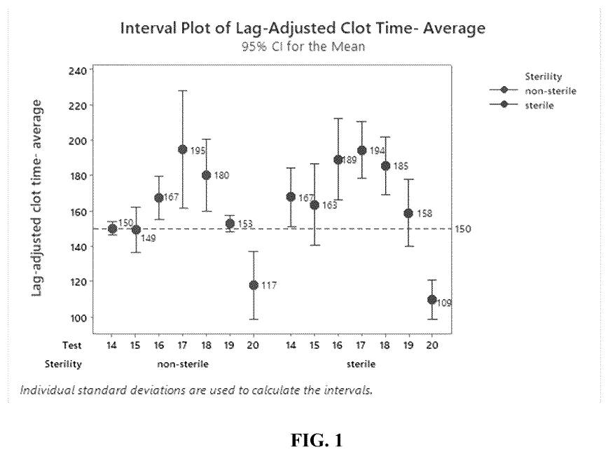

Samples were then tested in a Tilt Tube Clot Test consistent with Example 3. The results of the Tilt Tube Clot Test are summarized in FIG. 1. Clot test results show that the tested concentrations of CHA and CHD inhibit clotting, preventing samples from passing the clot time of less than 150 seconds. Gamma sterilization at maximum dose also appears to influence clot time, generally longer clot times for sterilized samples compared to non-sterile counterparts. In several instances, CHD at 300 μg/cm² and CHD at 050 μg/cm², these differences were statistically significant.

### Example 5

Using the procedures outlined in Example 2 and Example 3, substrates were prepared using the mixtures described in Table 11.

TABLE 11

| Test | Kaolin (g) | Glycerin(g) | CHX* (g) | DI Water (mL) |
|---|---|---|---|---|
| 21 | 0.52 | 1.40 | 0 | 60 |
| 22 | 0.52 | 1.40 | 0.01² | 60 |
| 23 | 0.52 | 1.40 | 0.005¹ | 60 |
| 24 | 0.52 | 1.40 | 0.001¹ | 60 |
| 25 | 1.04 | 2.81 | 0 | 60 |
| 26 | 1.04 | 2.81 | 0.01² | 60 |
| 27 | 1.04 | 2.81 | 0.005¹ | 60 |
| 28 | 1.04 | 2.81 | 0.001¹ | 60 |

* CHX = Chlorhexidine
¹Chlorhexidine diacetate (CHA)
²Chlorhexidine dihydrochloride (CHD)

The target concentrations of kaolin were 516.12 μg/cm² and 1032.34 μg/cm²; CHD concentrations were targeted at 10 μg/cm² and CHA concentrations were targeted at 5 and 1 μg/cm². It was noted during coating that the substrate used was different than the one used in Example 2 and absorbed around 10 mL of coating mixture on average rather than 12 mL. As a result, the % error was decreased.

The coat drying process was modified such that CHD/non-CHX samples were dried at 80 C for 2h, and CHA samples were dried at 54 C for four hours.

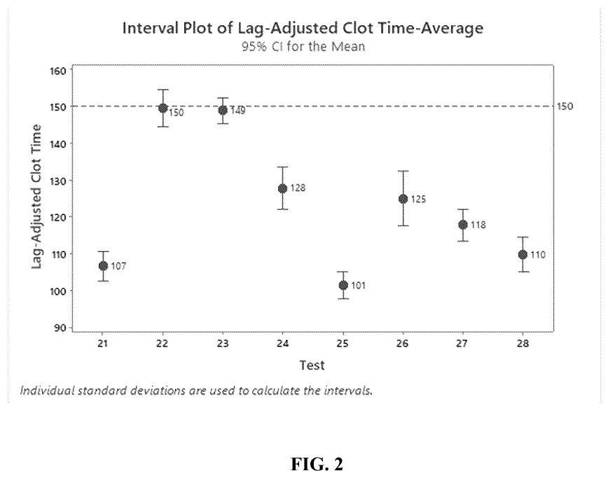

Samples were then tested in a Tilt Tube Clot Test consistent with Example 3. The results of the Tilt Tube Clot Test are summarized in FIG. 2. Clot test results show that increasing the kaolin by two times results in lower clot times even in the presence of CHX. Clot times were lowered by 20-40 seconds below the 150 second target which allows for a margin of safety when samples are sterilized and such that they can still pass the clot test.

### Example 6

Using the procedures outlined in Example 2 and Example 3, substrates were prepared using the mixtures described in Table 12.

TABLE 12

| | Kaolin (g) | Glycerin(g) | Anti-Microbial(g) | DI Water (mL) |
|---|---|---|---|---|
| 29 | 1.87 | 5.05 | 0.181² | 180 |
| 30 | 1.87 | 5.05 | 0.109² | 180 |
| 31 | 1.87 | 5.05 | 0.036² | 180 |
| 32 | 1.87 | 5.05 | 0.036¹ | 180 |
| 33 | 1.87 | 5.05 | 0.018¹ | 180 |
| 34 | 1.87 | 5.05 | 0.004¹ | 180 |
| 35 | 1.87 | 5.05 | 0 | 180 |
| 36 | 3.74 | 10.11 | 0.181² | 180 |
| 37 | 3.74 | 10.11 | 0.109² | 180 |
| 38 | 3.74 | 10.11 | 0.036² | 180 |
| 39 | 3.74 | 10.11 | 0.036¹ | 180 |
| 40 | 3.74 | 10.11 | 0.018¹ | 180 |
| 41 | 3.74 | 10.11 | 0.004¹ | 180 |
| 42 | 3.74 | 10.11 | 0 | 180 |
| 43 | 1.87 | 5.05 | 0.047³ | 180 |
| 44 | 1.87 | 5.05 | 0.109³ | 180 |

¹Chlorhexidine diacetate (CHA)
²Chlorhexidine dihydrochloride (CHD)
³Polyhexamethylene Biguanide (PHMB)

The target concentrations of kaolin were 516.12 μg/cm² and 1032.34 μg/cm²; CHD concentrations were targeted at 50, 30, and 10 μg/cm²; CHA concentrations were targeted at 10, 5, and 1 μg/cm²; and PHMB was targeted at two concentrations: 30 μg/cm² and 0.2% concentration by weight of the substrate, or approximately 13 μg/cm². Test 35 was a control without an anti-microbial agent.

For drying, non-antimicrobial, CHD, and PHMB samples were placed in an 80 C oven for 2 hours and for CHA a temperature of 54 C for 4 hours was used.

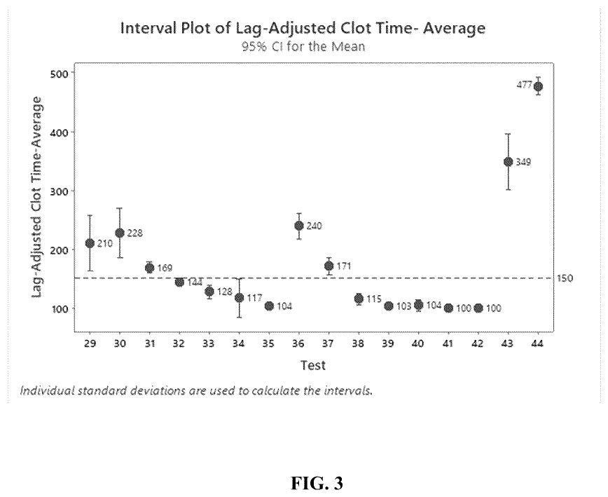

Samples were then tested in a Tilt Tube Clot Test consistent with Example 3. The results of the Tilt Tube Clot Test are summarized in FIG. 3.

Samples with chlorhexidine having a clot time of less than 150 seconds were sent to a contractor for HPLC analysis. Based on HPLC analysis, it was noticed that less chlorhexidine was present on the gauze than expected, and samples with higher kaolin tended to have less chlorhexidine. Results from the HPLC analysis are provided in Table 14.

TABLE 14

| | Avg | Theoretical | |
|---|---|---|---|
| | μg/cm² | μg/cm² | % of |
| | CHX | CHX | Theoretical |
| 38 | 3.53 | 10 | 35.3 |
| 39 | 3.57 | 10 | 35.7 |
| 40 | 0.42 | 5 | 8.4 |
| 41 | 0.00 | 1 | 0.0 |
| 32 | 5.88 | 10 | 58.8 |
| 33 | 2.02 | 5 | 40.4 |
| 34 | 0.06 | 1 | 6.0 |

CHX = chlorhexidine

### Example 7

Antimicrobial activity can be tested using an AATCC-100-2019 standard tested, or modification thereof.

1. An antimicrobial hemostatic device, comprising:
a substrate configured to be in contact with a bleed, said substrate comprising:
a hemostatic agent,
a biguanide based antimicrobial agent, or a pharmaceutically acceptable salt thereof, and
a binder configured to maintain the hemostatic agent with the substrate.

2. The device of claim 1, wherein the binder is glycerin.

3. The device of claim 1, wherein the hemostatic agent is kaolin.

4. The device of claim 1, wherein the biguanide based antimicrobial agent is chlorhexidine (preferably chlorhexidine diacetate or chlorhexidine dichloride) or polyhexamethylene biguanide (PHMB).

5. The device of claim 1, wherein said substrate further comprises an effective amount of a silver-based antimicrobial.

6. The device of claim 5, wherein the silver-based antimicrobial is colloidal silver, silver chloride, silver sulfadiazine, or silver nitrate.

7. The device of claim 1, wherein said substrate further comprises an effective amount of an antimicrobial iodine compound.

8. The device of claim 7, wherein the antimicrobial iodine compound is povidone-iodine or cadexomer iodine.

9. The device of claim 1, wherein a clot time for the device is less than 180 seconds.

10. The device of claim 9, wherein the clot time for the device is less than 150 seconds.

11. The device of claim 9, wherein a clot time for the device is between 100 and 150 seconds.

12. The device of claim 1, wherein a weight ratio of the biguanide based antimicrobial agent to the hemostatic agent is less than 0.04.

13. The device of claim 12, wherein the weight ratio is between 0.04 and 0.001.

14. The device of claim 13, wherein the weight ratio is between 0.02 and 0.001.

15. The device of claim 1, wherein the substrate is a textile.

16. The device of claim 15, wherein the textile is a gauze, preferably polyester-rayon.

17. The device of claim 1, wherein the biguanide based antimicrobial agent is present in an amount of 1 ug/cm² to 300 ug/cm², preferably 1 to 75, and most preferably 1 to 30.

18. The device of claim 1, wherein the substrate consists essentially of the hemostatic agent, the biguanide based antimicrobial agent, and the binder.

19. The device of claim 1, wherein the substrate consists of the hemostatic agent, the biguanide based antimicrobial agent, and the binder.

20. The device of claim 1, wherein the device at least one log reduction in growth compared to the untreated control, preferably greater than 2 log and most preferably greater than 4 log reduction in bacterial growth compared to an untreated (no antimicrobial agent) control.

21. A method of controlling or lessening the severity of bleeding, comprising contacting the substrate of the medical device of claim 1 with a bleed.

22. A method of preparing a device of claim 1, comprising:
combining the hemostatic agent, biguanide based antimicrobial agent, and binder in a liquid carrier to form a composition;
dipping or spraying the substrate in the composition; and
drying the substrate after the dipping or spraying, preferably wherein drying is at a temperature less than 80° C.

23. The method of claim 22, further comprising sterilizing the substrate after drying with gamma radiation.
